(19) United States
(12) Patent Application Publication
(10) Pub. No.: US 2003/0069504 A1
(43) Pub. Date: Apr. 10, 2003

(54) Receive filtering and filters for phase or amplitude coded pulse sequences

(75) Inventors: Wilko G. Wilkening, Bochum (DE); Helmut Ermert, Roettenbach (DE); Bernhard J. Brendel, Bochum (DE); Zuhua Mao, Issaquah, WA (US); Hui Jiang, Issaquah, WA (US)

Correspondence Address: Siemens Corporation, Intellectual Property Department, 186 Wood Avenue South, Iselin, NJ, 08830, US

(73) Assignee: Siemens Medical Solutions USA, Inc.

(21) Appl. No.: 10/197,289

(22) Filed: Jul. 16, 2002

Related U.S. Application Data
(60) Provisional application No. 60/327,484, filed on Oct. 5, 2001.

(51) Int. Cl. A61B 8/00

(52) U.S. Cl. 600/443

(57) ABSTRACT

Receive information for multiple pulse sequences are aligned as a function of phase shifts, amplitude weightings or other differences to account at least in part for inaccuracies in the transmit pulses, noise, focusing or other differences. Separate receive filters for each of the echo signals responsive to different transmit pulses provide frequency dependent amplitude weightings or phase shifts. The frequency dependent amplitude weightings or phase shifts compensate for imperfections in the transmit pulse prior to combining the echo signals. The filter may include a various number of taps or inputs, such as two or more taps, providing different spectral characteristics for different echo signals responses to the different transmit pulses. For example, N different linear receive filters are provided for echo signals responsive to each of N different transmit pulses, respectively. Any weightings for canceling information due to combination are applied to the corrected echo signals, and the weighted echo signals are combined to discriminate between nonlinear and linear information or different types of media (e.g. tissue, contrast agent, fluid, . . . ).

### RELATED APPLICATIONS

This application is a continuation-in-part of and claims the benefit of the filing date pursuant to 35 U.S.C. § 119(e) of Provisional Application Serial No. 60/327,484, filed Oct. 5, 2001, for a OPTIMIZED RECEIVE FILTERS AND PHASE-CODED PULSE SEQUENCES FOR CONTRAST AGENT AND NON-LINEAR IMAGING, the disclosure of which is hereby incorporated by reference.

### BACKGROUND

The present invention relates to ultrasonic imaging using phase or amplitude coded pulse sequences. In particular, discrimination of different imaged media or different spectral responses is provided.

Phase and/or amplitude coded pulse sequences have been used to distinguish media or scatterers having different nonlinear and linear propagation or scattering characteristics. Sequential transmit pulses and/or responsive signals are weighted or phase shifted for superposition to discriminate between nonlinear and linear information. For example, transmit and receive sequences and associated combination structures are disclosed in U.S. Pat. Nos. ______ and ______ (U.S. application Ser. Nos. 09/514,803 and 09/650,942, the disclosures of which are incorporated herein by reference. Other multiple pulse imaging techniques are disclosed in U.S. Pat. Nos. 5,951,478; 5,632,277; 6,095,980; 5,577,505 and 6,155,981). To form one line of image data, two or more echo signals resulting from a corresponding two or more different transmit pulses are acquired. The transmit pulses ideally have the same envelope and carrier frequency, but different carrier phases and/or amplitudes. The different echo signals are weighted and summed to cancel out undesired information. For example, information associated with linear scattering and propagation (e.g. fundamental transmitted frequencies) is cancelled. Depending on the carrier phases and amplitudes used, harmonics or subharmonic information is enhanced or passed. Different transmit and receive sequences, weightings and combinations result in canceling or reducing information at any of various frequencies or combinations of frequencies.

Commercial ultrasound systems may generate transmit pulses with different phases and/or amplitudes inaccurately. For example, 90 degree phase shifts for bipolar waveforms may be inexact. The suppression of undesired information based on the weighted superposition of receive signals responsive to the inaccurate transmit pulses is incomplete. Ultrasound systems generating other types of waveforms, including sinusoidal, or unipolar, may provide inaccurate phase shifts or amplitude interpulse differentiation.

### BRIEF SUMMARY

By way of introduction, the preferred embodiments described below include methods and systems for aligning receive information as a function of phase shifts or amplitude weightings to account at least in part for inaccuracies in the transmit pulses and/or focusing, possibly improving the signal-to-noise ratio or, in the case focusing, the point spread function. Desired and undesired information are differentiated based on the responses (echoes). The filters differentiate between two media, like contrast agent and tissue, to enhance the difference (e.g. harmonics) but also suppress whatever the two media have in common (e.g. linear scattering of both media and noise). Separate receive filters for the received signals responsive to different transmit pulses provide frequency dependent amplitude weightings or phase shifts. The frequency dependent amplitude weightings or phase shifts compensate for imperfections in the transmit pulses and perform additional processing to improve e.g. separation between two media, noise suppression or point spread function optimization prior to combining the echo signals. The receive filters may include a variable number of taps or inputs, such as two or more taps, providing different spectral characteristics for different echo signals responses to the different transmit pulses. For example, N different linear receive filters are provided for receive signals responsive to each of N different transmit pulses, respectively. Any weightings for canceling information due to combination are applied to the corrected receive signals, and the weighted receive signals are combined to discriminate between nonlinear and linear information or different types of media (e.g. tissue, contrast agent, fluid, . . . ).

The present invention is defined by the following claims, and nothing in this section should be taken as a limitation on those claims. Further aspects and advantages of the invention are discussed below in conjunction with the preferred embodiments.

### BRIEF DESCRIPTION OF SEVERAL VIEWS OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of an ultrasound system for generating information responsive to phase or amplitude coded pulse sequences.

FIG. 2 is a graphical representation of one embodiment of the phase spectra of filters for a four pulse sequence.

FIG. 3 is a graphical representation of one embodiment of amplitude spectra for a four pulse sequence.

FIGS. 4 and 5 are graphical representation of histograms representing discrimination between two different types of media.

FIG. 6 is a flow chart representing one embodiment of a transmit, receive and filtration sequences for discriminating between linear and nonlinear information.

### DETAILED DESCRIPTION OF THE PREFERRED EMBODIMENTS

Two levels of filtering or a transmit pulse specific filter and combination of information from multiple pulses are provided: weighted superposition of multiple receive signals and separately filtering one or more of the receive signals to compensate for imperfections in the transmit pulse and/or to optimize image information prior to the weighted superposition. Two different filtering stages are provided. As used herein, filtering includes weighing, weighted combinations, finite impulse response filters or other mathematical functions providing approximations of such functions. One specific embodiment utilizes these for phase shift or amplitude weighting.

Pulse sequences are adapted to discriminate different media based on nonlinear, frequency or focusing dependent interaction with the sound field. Transmit pulses are chosen so that the echoes that are returned from different media can reveal the differences between the media. In contrast agent imaging, these pulses are chosen to distinguish between tissue containing the contrast enhancing agent and tissue void of this agent. All pulse sequences discussed in U.S. Pat. Nos. 5,951,478; 5,632,277; 6,095,980; 6,155,981; 5,577,505 may be used. There is, however, no restriction to certain waveforms. The N transmit waveforms may be arbitrary or even identical. The transmit beamformer may also be used to modify the acoustic waves. The terms amplitude and phase coded do are used broadly, since both parameters may be chosen to be functions of time or other parameters.

In these implementations, the echoes resulting from a pulse sequence were superimposed by a weighted summation. A weighted summation is the same as the embodiment herein where the filter length is 1 (1 tap=1 filter coefficient=1 weight.). In order to cancel out the echoes from non-linear propagation and scattering, the transmit pulses have the same frequency response (magnitude spectra) and phases responses (phase spectra) except for a constant phase shift, which is e.g. 180 degrees for phase inversion. Pulses were designed to best meet these characteristics, and were also chosen to provide a good discrimination. Filters can correct for frequency dependent or other mismatches in the desired magnitude and phase spectra. This may be done without comparing the received spectra with the desired spectra. In one embodiment, the algorithms as described below do not primarily achieve this compensation. Instead, training data is used (i.e. acquire echoes for 2 or more media using the pulse sequences to calculate the filters) without consideration to how the transmitter does not reproduce the sequence accurately. Also, the 2 media do not have to be pure. For example, the two media are characterized as medium 1 and a mixture of media 1 and 2, such as medium 1 being tissue and medium 2 being contrast agent. A pure cancellation approach would try to cancel the echoes of medium one. This may not be exactly possible or difficult if nonlinear propagation is considered. In this case, even for exactly accurate transmit pulses, the contrast between the 2 media may be poor. The approach described herein designs filters that achieve the best discrimination. So, maybe the signals from tissue are not completely cancelled, but the contrast agent appears substantially brighter than the tissue. The filters suppress what the echoes from the 2 media have in common and enhance what is different. The filter, therefore suppress noise and also linear scattering from bubbles. The filters can also cope with inaccurate transmit signals as long as they are reproducible. Theoretically, any pulses may be used. The filters are based on the acquired training data or pre-determined characteristics. No knowledge about the design of the transmit pulses is needed. Some pulse sequences may provide better results than others. Short pulses and chirps may be combined within a sequence. Both types may have the same magnitude spectrum, but the short pulse compresses all frequencies in a short time and the chirp sweeps through the bandwidth. The filters, may recompress the chirp to a short pulse by introducing a frequency dependent delay. The chirp and the short pulse, assuming a similar or same total energy and bandwidth, may differ significantly in maximum amplitude and phase.

The N receive filters are determined so that after superposition of the filtered echo signals, the differences between two media are enhanced. The term “medium” may denote a physical medium and/or a location. To determine the filters, echo signals representing both media and all N transmit pulses are used. This training data consists of two sets of echo signals. Each set consists of at least echo signals that primarily, not necessarily exclusively, represent one of the two media for the N transmit pulses. The set of echo signals may consist of more echo signals to represent the medium more extensively, e.g. at different depth, acoustic power levels, concentrations etc. The training can be measured or simulated or partly measured and simulated. An algorithm determines the filters based on the training data.

The filters suppress noise because the echoes from both media have noise in common. The filters can compensate for inaccuracy in the transmit pulses, including nonlinearity of the system on the transmit side. Frequency dependent attenuation may be accounted for with the filters. In this case, training data for both media is acquired at different depths. Then, the filters are implemented as depth and maybe tissue-type dependent. Otherwise, it is preferred to acquire training data for different depths, but use the data to design one “average” set of filters for other regions. Filters may also be also transmit power and focus and line density dependent. Various filters and associated dependencies may be used, including filters that are not depth, tissue-type, power, focus, and/or line density dependent.

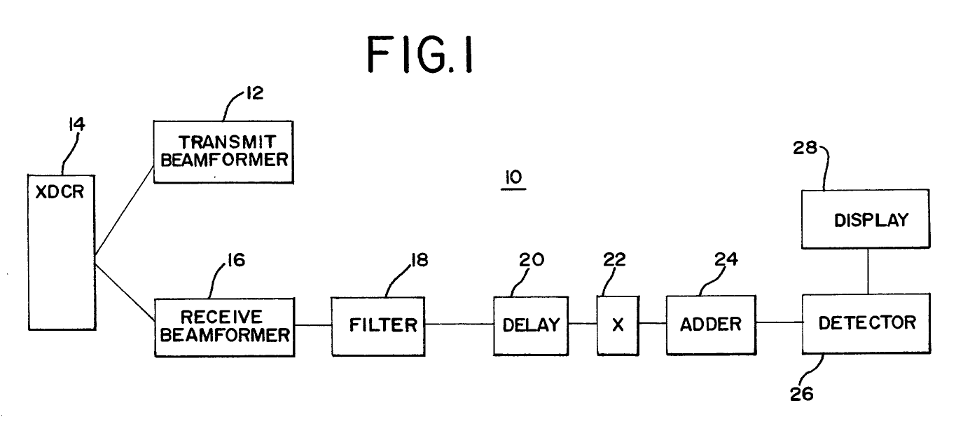

FIG. 1 shows one embodiment of a system 10 for generating information responsive to phase or amplitude coded pulse sequences. The phase and/or amplitude coded pulse sequences include phase or amplitude differences of transmit pulses or applied to receive pulses. The system 10 includes a transmit beamformer 12, a transducer 14, a receive beamformer 16, a filter 18, a delay 20, a multiplier 22, an adder 24, a detector 26 and a display 28. Fewer, different or additional components may be provided, such as including various delays, multipliers and/or additions in the filter, adding a scan converter, an additional detector, additional delays, additional filters, or a transmit and receive switch. In other embodiments, the order of the components is different, such as providing the delay 20 before the filter 18 or after the multiplier 22.

The system 10 uses amplitude and/or phase coded pulse sequences. For example, any of the transmit and receive sequences disclosed in U.S. Pat. Nos. ______ (U.S. application Ser. No. 09/514,803); ______ (U.S. application Ser. No. 09/650,942); 5,951,478; 5,632,277; 6,095,980; 6,155,981 and 5,577,505, the disclosures of which are incorporated herein by reference, are used. The transmit pulses preferably have a similar spectral energy distribution but different amplitudes and/or phase spectra. These sequences are associated with transmit pulses along a same or adjacent scan lines having different interpulse phase shifts and/or amplitudes. For example, the transmit and receive sequence includes two transmit pulses and two sets of associated receive signals responsive to each of the transmit pulses respectively. The transmit pulses have an opposite phase, such as 180 degree phase shift between transmit pulses. Other numbers of transmit pulses and associated receive signals, phase differences, or amplitude differences may be used. Phase shifts, amplitude weightings or combinations thereof may be applied to the receive signals, such as weighting one of three sets of receive signals responsive to three transmit pulses, respectively, with twice the amplitude weighting and an opposite phase than applied to other receive signals. The filtering and superposition (adding) then isolates spectral components representing the difference in the response of the two media.

The transmit beamformer 12 comprises analog and/or digital components, such as plurality of memories, delays and amplifiers for generating transmit waveforms for each element within a transmit aperture. Bi-polar, unipolar, sinusoidal or other waveforms are generated. The delays allow for phase adjustments to the transmitted waveforms so that the transmitted pulse generated by the transducer 14 is associated with a particular phase. The amplifiers control an amplitude of the transmitted pulse for apodization. Transmitted pulses are focused along a scan line. Multiple transmit pulses are sequentially formed along a same or adjacent scan lines applying identical or different focusing. The multiple pulses have different amplitudes or phasing. For example, three transmit pulses are sequentially fired along a same scan line with one transmit pulse having twice the amplitude of the other two pulses. As another example, four transmit pulses are transmitted along a same scan line with 90 degree phase differences (e.g., 0°, 90°, 180°, and 270°). As yet another example, multiple transmit pulses are formed along a same scan line having different interpulse amplitudes and phases.

The transducer 14 comprises a piezoelectric or microelectromechanical transducer of any dimension for generating mechanically or electrically focused transmit pulses along one or more scan lines in the media. The transducer 14 also receives echo signals. The echo signals are converted to electrical signals and provided to the receive beamformer 16.

The receive beamformer 16 comprises analog or digital components, such as (1) a plurality of amplifiers and delays forming channels for each of the elements in a receive aperture and (2) a summer for summing information from each of the channels to form beamformed signals. The beamformed signals represent information along the scan line of desired geometry. For example, a set of data representing spatial locations along a scan line is obtained using dynamic focusing of the receive beamformer 16. The beamformed signals comprise in-phase and quadrature information, but radio frequency or other data formats may be used. The beamformed signals are coherent, maintaining the phase information responsive to the transmitted pulse. The amplitude associated with the beamformed signals is responsive to the amplitude of the transmitted pulse.

The filter 18 comprises analog or digital components, such as a digital signal processor, application specific integrated circuit, memory buffers, multipliers, summer or other components for implementing a finite impulse response filter. In one embodiment, the filter is implemented as a multiplier or multiplication function, such as a one tap filter. Each input value is weighted by the multiplier. In other embodiments, two or more taps are provided. Using a delay or buffer, multiple beamformed signals representing the scan line are input to the filter 18. Two values responsive to a same transmit pulse and representing different spatial locations or the scan line as a function of time are input into the two taps. Each input is weighted by a filter coefficient and the weighted values are summed. The filter 18 filters beamformed signals responsive to one transmit pulse at any given time. The filter 18 may comprise one tap. In combination with the one tap multiplier 22, a two tap filter is provided prior to the combination of data by the adder 24. Other filter structures or operation, such as IIR (inifinite impulse response) or wave digital filters, including decimation filtering, may be used.

The filter coefficients or weights are programmable, allowing application of a different filter response for beamformed signals responsive to different transmit pulses. The filter 18 sequentially receives beamformed signals representing the scan line in response to sequential transmit pulses. The filter response is the same or different for each of the sets of beamformed signals. In other embodiments, the filter 18 comprises two or more separate filters. The separate filters have the same or different response for filtering sets of beamformed signals responsive to different transmit pulses. The filter 18 provides alignment of information for cancellation of undesired spectral components.

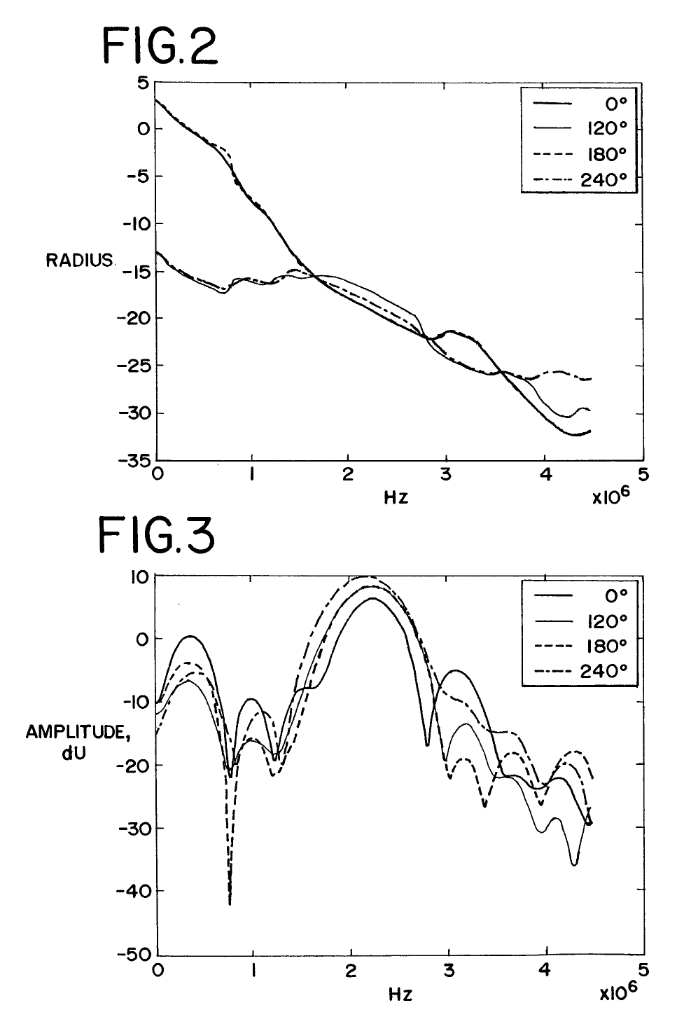

The different filter responses allows for frequency dependent phase shift and/or amplitude weighting. The spectral response of the filter 18 is selected as a function of the characteristic of the transmit pulses and of the media to be differentiated. Inaccuracies in generation of the transmit pulse are corrected by the frequency dependent response. Additional weighting and phasing is introduced to improve the differentiation of the media. The filters can also comprise additional filtering functionality to achieve noise reduction, pulse compression, resolution enhancement. The filter responses can be time variant to dynamically adapt the filter response to the imaging depth. Since transmit pulses have different phase or amplitude characteristics, different filter responses are provided for different sets of beamformed data. For example, FIG. 2 shows the phase spectral response of a 64 tap filter associated with transmission of four transmit pulses associated with 0 degree, 120 degree, 180 degree and 240 degree phasing. The frequency dependent phase spectra for echoes corresponding to the 0 and 180 degree carrier phases are similar since a bipolar transmitter is generally more accurate for 180 degree phase shifts, while other phase shifts require more shifting capabilities. Accordingly, the filters for the 120 degree and 240 degree phase shifts differ from the other filters and have less symmetry. FIG. 3 shows the amplitude spectra of the four 64 tap filters where the four transmit pulses are inaccurately transmitted with a desired same amplitude. The amplitude and phase spectral response of the filters varies as a function of frequency. The subharmonic frequency range and frequency range that matches the transducer bandwidth are emphasized in the beamformed signals while other frequency ranges tend to be suppressed, but other frequency relationships may result. The filters align the beamformed signals to correct for inaccuracies in the transmit pulses and to enhance contrast between the two media contrast agent and tissue mimicking material. Later combination of the beamformed signals responsive to different transmit pulses provides the desired spectral response enhancing the differences between the acoustical responses of the two media.

The frequency-dependent phase shift and amplitude weighting preferably enhance the contrast between two different types of media. For example, the filter coefficients are selected as a function of the energy ratio of beamformed data responsive to the two media and the same transmit pulse or transmit pulse sequence. Other energy ratios or variables may indicate contrast between two different media. Since the enhancement and suppression of harmonics is predominantly achieved by the summation due to the phase and amplitude relationships within the multiple pulse sequence, the resulting frequency ranges are not necessarily frequencies used for single transmit harmonic imaging. In one embodiment, the fundamental transmitted frequency range tends to be suppressed relative to other frequency ranges, providing better discrimination between two different media upon summation of beamformed signals responsive to different transmit pulses.

The delay 20 comprises one or more memories or buffers. The delay 20 delays a beamformed signal representing a spatial location in response to a first transmit pulse relative to a beamformed signal representing the same spatial location or depth responsive to a subsequent transmit pulse. The delay 20 provides beamformed signals responsive to different transmit pulses but a same or similar depth at a same time to the adder 25. In another embodiment, the delay 20, multiplier 22 and adder 24 comprise a filter, such as a finite impulse response filter implemented in a digital signal processor, application specific integrated circuit, analog components, digital components or combinations thereof. In one embodiment, the delay 20, multiplier 22 and adder 24 comprise a clutter filter of a Doppler processing path. The delay 20, multiplier 22 and the adder 24 operate as discussed in the patents above to discriminate linear and nonlinear or different types of media for detection and imaging.

The multiplier 22 comprises a plurality of digital or analog multipliers for weighting beamformed signals. The beamformed signals responsive to one transmit pulse are weighted relative to beamform signals responsive to a different transmit pulse. In one embodiment, one or more multipliers for beamformed signals comprise a signal line for passing beamformed signals without multiplication, such as for applying a coefficient value of 1. For example, two sets of data responsive to two transmit pulses of a three transmit pulse sequence are weighted with a one-half or one value or coefficient. The third set of beamformed data responsive to the third transmit pulse is weighted with a one or two value. Other relative weightings may be used. In alternative embodiments, the multiplier 22 is positioned before the delay 20 or before the filter 18.

The adder 24 comprises analog or digital components for summing, subtracting or otherwise combining two or more inputs. For example, weighted beamformed signals are added. In one embodiment, the adder 24 includes one or more inverters or an inverted input for implementing a subtraction function. For example, three receive beamformed signals with 1, 2, 1 or ¼, ½, ¼ weighting and representing a same depth or spatial location are input to the adder 24. The beamformed signal associated with the greater weighting is inverted (e.g., [1-2 1]). The adder 24 outputs a value representing the combination of the sum of the lesser weighted beamform signals subtracted from the greater rate of beamform signals. Other weightings and inversions or additional schemes may be used.

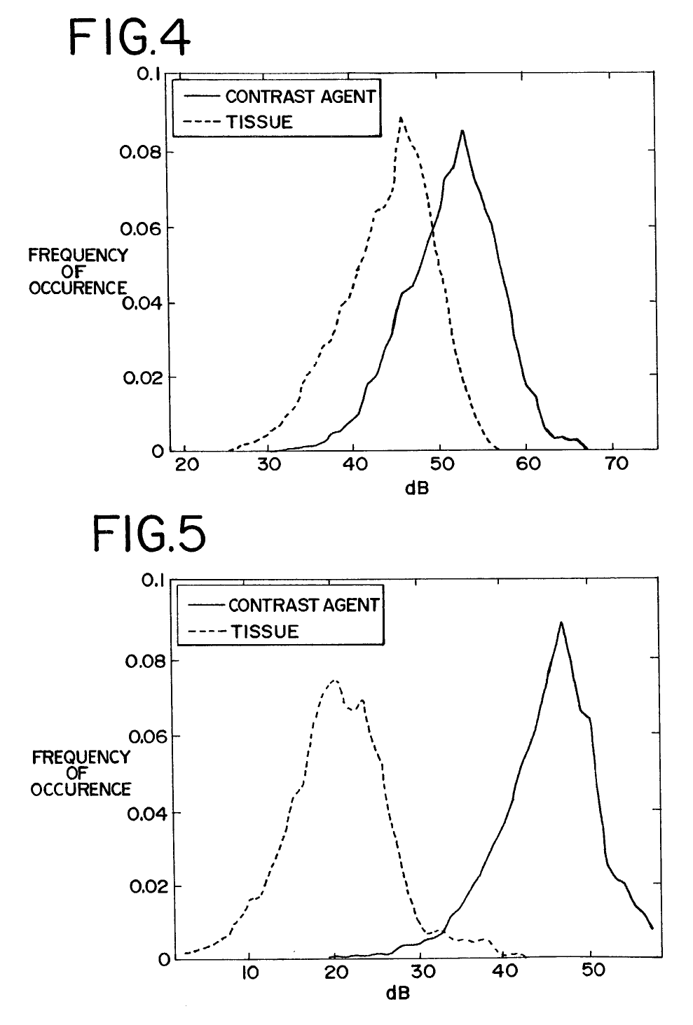

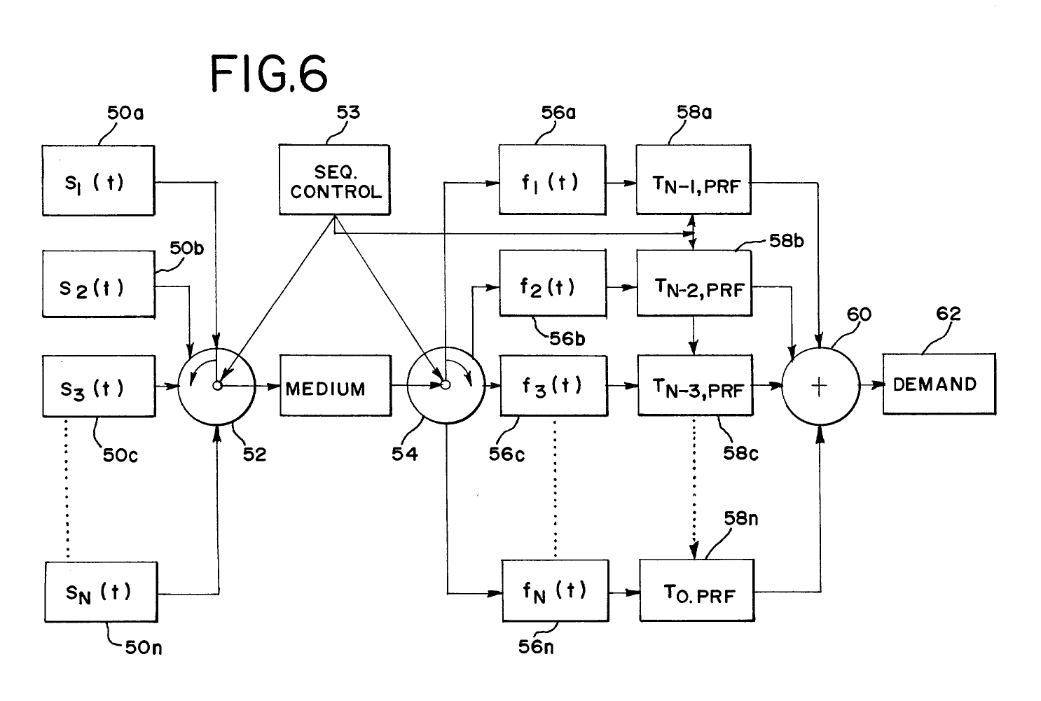

As discussed in the patents referenced above, adding beamformed signals responsive to the different transmit pulses discriminates between linear and non-linear information. By various combinations of weighting, combining, and phase or amplitude differences, information at different frequency bands is isolated, such as isolating odd harmonics, even harmonics, second harmonic, subharmonic or fundamental frequency information. Information at other frequencies' is reduced or removed by the combination of the information responsive to different transmit pulses. Undesired and desired spectral components may also share frequency bands but can nevertheless be isolated from one another because of the nonlinear response of one or both of the media to the amplitude and/or phase coding. Different media may be associated with different spectral responses. For example, contrast agent information is discriminated from tissue information. One example of discriminating between tissue and contrast agent information is provided using the transmit sequence and filter responses discussed above for FIGS. 2 and 3. A fundamental transmit frequency of 2.0 Megahertz is generated using a 3.5 megahertz curved array with four transmit pulses with relative phasing of 0 degrees, 120 degrees, 180 degrees, and 240 degrees. FIG. 4 shows a normalized histogram of contrast agent and tissue information responsive to the combination of beamform signals with the same weighting using addition (e.g., [1 1 1 1]). Due to the inaccuracies of the transmit pulses and the nature of the tissue and contrast agent, some of the contrast agent and tissue information overlap. FIG. 4 represents the combination of information without the filtering provided by the filter 18. FIG. 5 shows a normalized histograms for the representation of the contrast agent and tissue using the filters 18 with the filter responses shown in FIGS. 2 and 3. The overlap of the contrast agent and tissue information is minimized by the frequency dependent amplitude weighting and phase shift provided by the filter 18. By providing a threshold at 33 dB, a classification error of less than 3.5 percent in the depth range of the sample regions of a B-mode image is provided. Less or more discrimination between frequency ranges or types of media may be provided, such as using a one tap filter 18 with minimal or no frequency dependent adjustment. Such one tap filters still provide further discrimination between the different media.

The detector 26 comprises a B-mode detector, a Doppler detector, an amplitude detector or other detector of information from the coherently combined beamformed signal output by the adder 24. For example, the detector 26 comprises a Doppler energy detector. As another example, the detector 26 outputs B-mode detected intensity values. The detected information is formatted and used to generate an image on the display 28. Since undesired information is removed, the detector 26 detects desired spectral components, such as the nonlinear second harmonic frequency or a fundamental frequency without clutter or noise caused by information at other frequencies. The greater discrimination between frequency and phase information provided by the filters 18 results in an image that is more free of clutter or noise and allows better identification of media of interest.

FIG. 6 shows one embodiment of a process for imaging using optimized receive filters with pulse sequences. One through N transmit pulses, S_{N}(t), are generated in acts 50a-N. As represented in act 52, one or more of the transmit pulses S_{N}(t) is associated with a different phase and/or amplitude. The transmit pulses propagate through a medium, such as tissue and contrast agent of a patient. In act 54, receive beamformed signals are generated in response to each of the transmit pulses. In act 53, the sequence of transmit pulses and associated formation of responsive beamformed data is controlled. In acts 56a-n, beamform signals responsive to each of the transmit pulses are separately filtered. The filtered beamformed signals are then delayed in acts 58a-n to temporarily align information representing the same depths. In act 60, beamformed signals responsive to different transmit pulses are weighted and combined or just combined without further weighting. In act 62, the combined information is demodulated or detected. Additional, different or fewer acts may be provided, such as using only two transmit pulses and associated sets of beamformed data.

The acts of FIG. 6 are based on either of an optimal suppression of fundamental frequency information or an optimal echo energy ratio between signals from two media. Due to various nonlinear effects, the optimal echo energy ratio is preferably used as in the discussion below. Other design criteria may be optimized, such as suppression of even harmonics. Since convolution and summation are linear operations, determining the optimal energy ratio for two media can be described and solved by linear algebra.

The transmit pulses of acts 50a-n are represented as

sᵢ₀ (t)=aᵢ·g(t)·cos(ω₀t+φᵢ), i=1 . . . N, (1)

where aᵢ is, amplitude, ω₀ is carrier frequency, and φᵢ∈R the carrier phase shift. This representation is altered to account for system inherent errors:

sᵢ₀(t)=aᵢ·gᵢ(t)·cos(ωᵢt+φᵢ), (2)

where gᵢ(t)≈g(t), ωᵢ≈ω₀.

To allow for echo signals to return from the maximum imaging depth before transmission of the next sequential transmit pulse, a delay time, T_{PRI}, is introduced between each transmit pulse. An additional delay to avoid reverberation artifacts may also be included. The entire transmit pulse sequence is then represented by:

$${s}_{0} ( t ) = {\sum }_{i = 1}^{N} {s}_{i0} ( t - ( i - 1 ) \cdot {T}_{P R F} ) . \qquad ( 3 )$$

Any of the transmit pulses may have a different amplitude and/or phase as compared to other of the transmit pulses. Nonlinear distortion of the programmed pulses up to the electro-acoustic conversion, such as due to asymmetry of bipolar waveforms, is included within the representation of the transmit pulse sequence, s₀(t). The linear response of transmit side by the transmit beamformer and transducer, such as the frequency response of the transducer, is described by a linear impulse response h_{0T}. Accounting also for the linear impulse response, each transmit pulse sequence is described by:

$$s ( t ) = {\sum }_{i = 1}^{N} {s}_{i} ( t - ( i - 1 ) \cdot {T}_{P R F} ) , \qquad ( 4 )$$

where sᵢ(t)=sᵢ₀(t)*h_{0T}(t).

In act 52, the transmit pulses within the transmit pulse sequence are sequentially transmitted into the medium or patient. Echo signals scattered within the medium are then received by the transducer.

In act 54, beamformed signals e(t) are generated in response to the echo signals. The beamformed signals reflect the delay time T_{PRI} as given by:

$${e}_{0} ( t ) = {\sum }_{i = 1}^{N} {e}_{i0} ( t - ( i - 1 ) \cdot {T}_{P R F} ) . \qquad ( 5 )$$

The linear impulse response for receiving echo signals and generating beamformed signals is represented by h_{0R}, resulting in:

$$e ( t ) = {\sum }_{i = 1}^{N} {e}_{i} ( t - ( i - 1 ) \cdot {T}_{P R F} ) , \qquad ( 6 )$$

where eᵢ(t)=eᵢ₀(t)*h_{0R}(t).

After forming the beamformed signals as sets of data representing spatial locations along a scan line, the time axis is adjusted so that echoes appear to be simultaneous regardless of the time delay, T_{PRI}. The delay 20 (FIG. 1) provides the time adjustment in act 58a-n.

The linear propagation and reflection or scattering is characterized by an impulse response q(t). The resulting echo or beamformed response is provided by:

eᵢ₀(t)=sᵢ(t)*q(t) (7)

In acts 56a-n, the beamformed datasets are filtered. Fewer filters than associated transmit pulses may be used. The filtration and combination of acts 56 and 60 are represented as a convolution of N different filters assigned to N sets of beamformed data and a summation of the convolution as represented by:

$$r ( t ) = {\sum }_{i = 1}^{N} {e}_{i} ( t ) * {f}_{i} ( t ) . \qquad ( 8 )$$

Given ideal transmit pulses with different phases or amplitudes, filters with a constant or non-zero frequency response can be found so that the convolution, r(t), is equal to zero for each and every time. For more practical implementation, the filtering acts 56a-n limit the bandwidth of the beamformed signals to provide a convolution response of zero over a limited frequency range or frequency bands. For a nonlinear medium, the summation results r(t) is generally non zero. Nonlinear scatterers and propagation is detected by outputting the non-zero result of summation for detection. In alternative embodiments, the transmit and receive sequences and associated combination cause the result to be non-zero for different frequency bands, such as the fundamental frequency band or odd harmonic frequencies including the fundamental frequency band and substantially zero for other frequency bands.

The filtering acts 56a-n represent sequential filtering by a same filter or parallel filtering by multiple filters. The filter response of each of the acts 56a-n is the same or different, such as providing frequency dependent amplitude weighting and/or phase shifting. The filter coefficients are selected based on the desired results.

For contrast agent imaging or nonlinear tissue imaging, two different media are discriminated from each other. Both media may be described mathematically by nonlinear impulse responses q₁(s(t)) and q₂(s(t)). For any given spatial location, the beamformed data includes information about the scattering of the medium at that location as well as propagation of energy between the transducer and the spatial location. Assuming the nonlinear scattering is more substantial than nonlinear propagation, the two media are compared within the same depth range. The transmit path through any medium, linear or nonlinear, between the transducer and a particular spatial location provides a modified excitation signal for this depth. Nonlinear propagation on the receive path is negligible due to low amplitude after scattering. The linear propagation is included as discussed above in h_{0R}(t) of equation 6 above.

The energy ratio between two beamformed, filtered, and superimposed receive signals are associated with different M₁ and M₂. For example, an image is acquired having regions representing tissue and regions representing contrast agent, roughly at the same depth range. To improve the robustness of the optimization, a greater amount of data is acquired. Several scan lines K are acquired for each of the 2 media. In equation (12), the integral over t describes some kind of averaging over the depth range, such as 4-5 cm. Assume a region represents tissue in the depth range with a lateral extension ranging for 1.5 to 2.5 cm in the image. If the line width is 0.05 cm, 20 lines cover that lateral range. The training data for tissue is K=20 lines, where each line is represented by N echoes corresponding to the N transmit pulses. The vector length (length in samples) of each echo is defined by the depth range, the sampling frequency and the speed of sound. The two sets of echoes serving as training data may represent different depth and depth ranges, and they may also have different numbers of lines.

In practice, the training data sets may be cut and pasted together from different acquisitions, such as experimentally determining filter optimizations for programming systems prior to any particular imaging session. Alternatively, in a clinical situation, adaptive or real-time training may be used. First, the radio frequency of baseband data is stored in the system. For most contrast agent exams, some wash-in/wash-out process is observed. This means that frames with and without contrast agents are in the memory. This can be used to first train or optimize the filters. Then, all the stored data can be reprocessed or newly acquired data can be processed with the trained filters to achieve improved contrast visualization. An optimized set of filters fᵢ enhances the image contrast between the media. Equation 8 is solved for both media, as represented by:

$$\begin{matrix}{}^{1} r ( t ) = {\sum }_{i = 1}^{N} {}_{}^{1}{e}{}_{i}^{} ( t ) * {f}_{i} ( t ) & receive signal , {M}_{1} , \\ {}^{2} r ( t ) = {\sum }_{i = 1}^{N} {}_{}^{2}{e}{}_{i}^{} ( t ) * {f}_{i} ( t ) & receive signal , {M}_{2} .\end{matrix} \qquad ( 10 )$$

The energy ratio of two or more beamformed receive signals provides a measure of the image contrast as represented by:

$$c = \frac{{\int }_{t} {[ {\sum }_{i = 1}^{N} {}_{}^{1}{e}{}_{i}^{} ( t ) * {f}_{i} ( t ) ]}^{2} ⅆ t}{{\int }_{t} {[ {\sum }_{i = 1}^{N} {}_{}^{2}{e}{}_{i}^{} ( t ) * {f}_{i} ( t ) ]}^{2} ⅆ t} . \qquad ( 11 )$$

An energy ratio based on beamformed signals received along a single scan line in response to a same or different transmit pulses may be used, but information associated with a plurality of scan lines may better represent the acoustic properties of the different media. For example, beamformed signals for K₁ lines associated with the medium M₁ and K₂ scan lines associated with the medium M₂ are acquired. The contrast is then represented by:

$$c = \frac{\frac{1}{{K}_{1}} {\sum }_{k = 1}^{{K}_{1}} {\int }_{t} {[ {\sum }_{i = 1}^{N} {}_{k}^{1}{e}{}_{i}^{} ( t ) * {f}_{i} ( t ) ]}^{2} ⅆ t}{\frac{1}{{K}_{2}} {\sum }_{k = 1}^{{K}_{2}} {\int }_{t} {[ {\sum }_{i = 1}^{N} {}_{k}^{2}{e}{}_{i}^{} ( t ) * {f}_{i} ( t ) ]}^{2} ⅆ t} \qquad ( 12 )$$

Different media may be identified by application of a contrast threshold. One type of media is above the threshold and another type is below a threshold. Threshold ranges for distinguishing two or more media may be used. To identify specific types of media, the algorithms or equations may be adjusted or the filter optimized such that maximum and minimum contrast result for regions most like two media being distinguished.

The above representation may be converted into the discrete time domain as:

t=l·T, l∈ [symbol] , T∈ [symbol] ⁺, (13)

where T is the sampling interval. The K scan lines corresponding to a medium M cover the same depth range in the time range L·T. The index l for the minimal depth is defined as zero. The length of the filters is set to J taps. A convolution of the signal L samples with a J tap filter provides as an output L+J−1 samples. The contrast of equation 12 is mathematically represented in the discrete time domain as:

$$c = \frac{\frac{1}{{K}_{1}} {\sum }_{k = 1}^{{K}_{1}} {\sum }_{l = 0}^{L + J - 2} {[ {\sum }_{i = 1}^{N} {}_{k}^{1}{e}{}_{i}^{} ( l \cdot T ) * {f}_{i} ( l \cdot T ) ]}^{2}}{\frac{1}{{K}_{1}} {\sum }_{k = 1}^{{K}_{2}} {\sum }_{l = 0}^{L + J - 2} {[ {\sum }_{i = 1}^{N} {}_{k}^{2}{e}{}_{i}^{} ( l \cdot T ) * {f}_{i} ( l \cdot T ) ]}^{2}} , \qquad ( 14 )$$

where in any expression [s(l·T)]², the samples or vector components are squared.

The convolution in equation 14 is represented as a multiplication of a matrix with a vector as:

eᵢ(l·T)*fᵢ(l·T)=Εᵢ·fᵢ,

fᵢ=(f_{i,0} f_{i,1} . . . f_{i,J-1})^{T},

f_{i,l}=fᵢ(l·T) (15)

Simplifying the formulation of the summation provides:

$${\sum }_{i = 1}^{N} {}_{k}^{}{e}{}_{i}^{} ( l \cdot T ) * {f}_{i} ( l \cdot T ) = E \cdot f , \\ E = [ \begin{matrix}{E}_{1} & {E}_{2} & ⋯ & {E}_{N}\end{matrix} ] , \\ f = {( \begin{matrix}{f}_{1}^{T} & {f}_{2}^{T} & ⋯ & {f}_{N}^{T}\end{matrix} )}^{T} . \qquad ( 16 )$$

The energy of a time-discrete receive signal r(l·T) is expressed in

$$\begin{matrix}\vert r {\vert}^{2} = T \cdot {\sum }_{l = 0}^{L + J - 2} {[ {\sum }_{i = 1}^{N} {e}_{i} ( l \cdot T ) * {f}_{i} ( l \cdot T ) ]}^{2} \\ = T \cdot {f}^{T} \cdot {E}^{T} \cdot E \cdot f , \\ r = {( \begin{matrix}{r}_{0} & {r}_{1} & ⋯ & {r}_{L + J - 2}\end{matrix} )}^{T} , {r}_{l} = r ( l \cdot T ) .\end{matrix} \qquad ( 17 )$$

The average energy resulting from K beam lines equals

$$\frac{T}{K} \cdot {\sum }_{k = 1}^{K} {\sum }_{l = 0}^{L + J - 2} {[ {\sum }_{i = 1}^{N} {}_{k}^{}{e}{}_{i}^{} ( l \cdot T ) * {f}_{i} ( l \cdot T ) ]}^{2} = \frac{T}{K} \cdot {\sum }_{k = 1}^{K} {f}^{Γ} \cdot {}_{k}^{}{E}{}_{}^{T} \cdot {}_{k} E \cdot f = T \cdot {f}^{T} \cdot {E}^{′} \cdot f , \\ {E}^{′} = \frac{1}{K} {\sum }_{k = 1}^{K} {}_{k}^{}{E}{}_{}^{T} \cdot {}_{k} E . \qquad ( 18 )$$

Equation 14 can be rewritten as:

$$c = \frac{{f}^{T} \cdot {}_{}^{1}{E}{}_{}^{′} \cdot f}{{f}^{T} \cdot {}_{}^{2}{E}{}_{}^{′} \cdot f} \qquad ( 19 )$$

To optimize the contrast c as a function of the N filters represented in f, the straightforward approach, i.e. calculation the first derivative of c, leads to a nonlinear equation system with N·J equations. The filters in f are constrained to fulfill the following condition:

f^{T}·²Ε′·f=1. (20)

Since equation 19 is invariant with respect to a scaling of f, the normalization of equation 20 is possible. Combining equations 19 and 20 yields

c=f^{T}·¹Ε′·f (21)

The filters f that maximize c with the constraint of equation 20 are determined. The optimization problem can then be solved by means of Lagrange multipliers. The optimized function is:

f^{T}·¹Ε′·f+λ·(f^{T}·²Ε′·f−1). (22)

Using the derivative of equation 22, the solution for f can be found by the following equation system:

¹Ε′·f+λ·²Ε′·f=0 (23)

where equation 23 represents a generalized Eigenvalue problem. Since ²Ε′ is invertible, a left multiplication of the equation 23 by the inverse of ²Ε′ leads to the traditional Eigenvalue problem:

(²Ε′)⁻¹·¹Ε′·f+λ·f=0 (24)

The Eigenvectors contain filter coefficients of all N filters. In total, N·J complete sets of filters f are represented in the Eigenvectors. After scaling the Eigenvectors to fulfill equation 20, the Eigenvector that maximizes the contrast c is determined by evaluating equation 21. The components of that Eigenvector are the filter coefficients for the N filters, where each filters has J coefficients. It is not required to use the Eigenvector, i.e. the set of filters, that provides the highest contrast as given by c. Other sets of filters may provide less contrast but better axial resolution. For some applications, it is useful to select several sets of filters with a high and similar contrast c. For each of these sets of filters, an image is derived by demodulation (envelope detection, etc.). These images will have similar contrast. Since the images are derived from different filters and, therefore, form different spectral components, the speckle pattern is partly different. The histograms as given in FIG. 5 may be similar for these images, i.e. the histograms representing the two media have an equivalent mean brightness and an equivalent variance (width of the distribution). The spatial distribution of the intensities represented in the histograms differ between the images. Therefore, averaging the images reduces the variation in intensity for both media. The variance (width) of the histograms is reduced, while the means of the histograms are more or less unchanged, reducing the overlap between the histograms and improving the separation of the two media. In alternative embodiments, the filter coefficients are determined through experimentation or are preset without using any feedback or adaptation based on beamformed signals. For example, different filter coefficients, i.e. different sets of filters, are provided for different imaging applications in response to user configuration of the ultrasound system 10.

In act 60, the filtered and delayed beamformed data is weighted. In alternative embodiments, one or all of the beamformed data combined is free of additional weighting. The beamformed data is then combined, such as by subtraction or addition. Other mathematical functions for combination including linear or nonlinear combination may be provided. The combination discriminates between different spectral components or different types of media. The output includes information for desired spectral components or media with a reduction of information associated with undesired spectral components or media.

In act 62, the output information is demodulated or detected. The detected information is used to generate an image of the desired information, such as information at the desired spectral components or of the desired media.

Various alternative transmit and receive sequences may be used. U.S. Pat. No. 6,155,981, the disclosure of which is incorporated herein by reference, discloses transmit and receive pulse sequences that may be used. Filtering may be provided by mere phase shifting or amplitude weighting alone. In one embodiment, four sequential transmit pulses and associated sets of beamformed data are generated. A same amplitude is used on transmit with 90 degree phase differences of a 45 degree phase, 145 degree phase, 225 degree phase and 315 degree phase. Uniform weighting is applied to the associated beamformed signals prior to combination. The pre-filtering of the filter 18 or different filtering for different sets of beamformed data responsive to the different transmit pulses accounts for phase shift errors in the transmit beamformer 12. As another example, a 7.2 Megahertz linear array is used to transmit a pulse sequence of five pulses at a carrier frequency of 6 Megahertz. Each of the five transmit pulses is associated with a phase that differs from the preceding transmit pulse by 72 degrees. Beamformed signals responsive to each of the five transmit pulses are separately filtered in response to different spectral characteristics. The filtered signals are summed and detected to represent a scan line in an image. The filter may limit the frequency range of subharmonics, such as 0 to 2 Megahertz range. For imaging contrast agents, the frequency of operation and associated shift are selected to correspond to the response characteristic of any contrast agent used. The discrimination between different types of media or different frequency ranges is based on the scattering response of the insonified tissue or contrast agent.

While the invention has been described above by reference to various embodiments, it should be understood that many changes and modifications can be made without departing from the scope of the invention. For example, any two media that differ in terms of nonlinearity or frequency dependent back scattering or attenuation may be differentiated using filter optimization. As mentioned above, the two media may also represent different locations so that the same filters may be optimized to concentrate energy in a certain point with respect to the surrounding, thus improving the point spread function for multiple transmits per line (multiple focal zones). It is therefore intended that the foregoing detailed description be understood as an illustration of the presently preferred embodiments of the invention, and not as a definition of the invention. It is only the following claims, including all equivalents, that are intended to define the scope of the invention.

What is claimed is:

1. A method for generating information responsive to pulse sequences, the method comprising:
(a) combining of first and second beamformed signals responsive to sequential first and second transmit pulses;
(b) separately filtering at least one of the first and second beamformed signals prior to (a), at least one filter including at least two taps, where filtering applied to the first beamformed signal is different than filtering applied to the second beamformed signal.

2. The method of claim 1 wherein (b) comprises applying a set of data responsive to the first transmit pulses to the at least two taps.

3. The method of claim 1 further comprising:
(c) filtering the second beamformed signal, the filtering of (c) having a different spectral response than the filtering of (b).

4. The method of claim 1 wherein (b) comprises providing at least one of a frequency dependent amplitude weighting and frequency dependent phase shift.

5. The method of claim 1 wherein (a) comprises adding the first and second beamformed signals wherein the first transmit pulse has a different phase than the second transmit pulse, wherein (a) discriminates between linear and non-linear information, and further comprising:
(c) detecting the non-linear information.

6. The method of claim 1 further comprising:
(c) weighting the first beamformed signal after (b) and prior to (a).

7. The method of claim 6 further comprising:
(d) weighting the second beamformed signal with a different weight than used in (c).

8. The method of claim 1 wherein (a) comprises adding the first and second beamformed signals wherein the first transmit pulse has a different phase and amplitude than the second transmit pulse.

9. The method of claim 1 further comprising:
(c) sequentially transmitting the first and second transmit pulses along a scan line, the first transmit pulse having a 45 degree phase shift and the second transmit pulse having a 135 degree phase shift; and
(d) sequentially transmitting third and fourth transmit pulses along the scan line, the third transmit pulse having a 225 degree phase shift and the fourth transmit pulse having a 315 degree phase shift;
wherein (a) comprises adding the first and second beamformed signals with third and fourth beamformed signals, the third and fourth beamformed signals responsive to the third and fourth transmit pulses, respectively.

10. The method of claim 1 further comprising:
(c) sequentially transmitting the first and second transmit pulses along a scan line, the first and second transmit pulses each corresponding to bi-polar transmit waveforms.

11. A system for generating information responsive to pulse sequences, the system comprising:
a receive beamformer for generating first and second beamformed signals responsive to sequential first and second transmit pulses, respectively;
an adder for combining of the first and second beamformed signals; and
a filter connected between the beamformer and the adder, the filter operable to filter the first beamformed signals, the filter including at least two taps where the filter applied to the first beamformed signal is different than filtering applied to the second beamformed signal.

12. The system of claim 11 wherein the first beamformed signals comprise data representing different depths along a scan line and the filter is operable to receive at least two of the first beamformed signals at the at least two taps, respectively.

13. The system of claim 11 further comprising:
a delay connected between the receive beamformer and the adder, the delay operable to delay application of the first beamformed signals to the adder, wherein the filter is operable to sequentially filter the second beamformed signals and the first beamformed signals.

14. The system of claim 11 wherein the filter provides at least one of a frequency dependent amplitude weighting and frequency dependent phase shift to the first beamformed signals.

15. The system of claim 11 further comprising:
a detector operable to detect one of non-linear and linear information output by the adder, wherein the adder is operable to discriminate between non-linear and linear information based on different relative phases or amplitudes of the first and second transmit pulses.

16. The system of claim 11 further comprising:
a weighting multiplier operable to weight first beamformed signals output by the filter, wherein the adder receives the output of the weighting multiplier.

17. A method for generating information responsive to phase or amplitude coded pulse sequences, the method comprising:
(a) combining of first and second beamformed signals responsive to sequential first and second transmit pulses, respectively;
(b) discriminating between two media based on (a);
(c) filtering the first beamformed signal prior to (a) in response to a first spectral response with at least two taps; and
(d) filtering the second beamformed signal prior to (a) in response to a second spectral response, the second spectral response different than the first spectral response.

18. The method of claim 17 wherein (c) comprises filtering with the at least two taps, the first beamformed signal applied to one of the at least two taps and another beamformed signal responsive to the first transmit pulse and associated with a different depth applied to the other of the at least two taps.

19. The method of claim 17 wherein (c) comprises providing at least one of a frequency dependent amplitude weighting and frequency dependent phase shift.

20. The method of claim 17 further comprising:
(e) weighting the first beamformed signal after (c) and prior to (a).

21. The method of claim 20 further comprising:
(f) weighting the second beamformed signal after (d) with a different weight than used in (e).

22. The method of claim 17 further comprising:
(d) sequentially transmitting the first and second transmit pulses along a scan line, the first transmit pulse having a 45 degree phase shift and the second transmit pulse having a 135 degree phase shift; and
(e) sequentially transmitting third and fourth transmit pulses along the scan line, the third transmit pulse having a 225 degree phase shift and the fourth transmit pulse having a 315 degree phase shift;
wherein (a) comprises adding the first and second beamformed signals with third and fourth beamformed signals, the third and fourth beamformed signals responsive to the third and fourth transmit pulses, respectively.

23. A system for generating information responsive to phase or amplitude coded pulse sequences, the system comprising:
a receive beamformer for generating first and second beamformed signals responsive to sequential first and second transmit pulses, respectively;
an adder for combining of the first and second beamformed signals; and
an at least two tap filter connected between the beamformer and the adder, the filter operable to filter the first and second beamformed signals in response to first and second spectral responses, respectively, the second spectral response different than the first spectral response.

24. The system of claim 23 wherein the first beamformed signals comprise data representing different depths along a scan line, where the filter is operable to receive at least two of the first beamformed signals at the at least two taps, respectively.

25. The system of claim 23 further comprising:
a delay connected between the receive beamformer and the adder, the delay operable to delay application of the first beamformed signals to the adder, wherein the filter is operable to sequentially filter the second beamformed signals and the first beamformed signals.

26. The system of claim 23 wherein the filter provides at least one of a frequency dependent amplitude weighting and frequency dependent phase shift to the first and second beamformed signals.

27. The system of claim 23 further comprising:
a weighting multiplier operable to weight first beamformed signal output by the filter, wherein the adder receives the output of the weighting multiplier.

28. A method for generating information responsive to phase or amplitude coded pulse sequences, the method comprising:
(a) combining of first and second beamformed signals responsive to sequential first and second transmit pulses, respectively, the first transmit pulse having one of a different phase and amplitude than the second transmit pulse;
(b) discriminating between non-linear and linear information based on (a);
(c) filtering the first beamformed signal prior to (a); and
(d) providing at least one of a frequency dependent amplitude weighting and frequency dependent phase shift based on (c).

29. The method of claim 28 wherein (c) comprises filtering with a spectral response that is a function of characteristics of the first and second transmit pulses.

30. The method of claim 29 wherein (c) comprises filtering with a spectral response that is a function of an energy ratio of the first and second beamformed signals.

31. A system for generating information responsive to phase or amplitude coded pulse sequences, the system comprising:
a receive beamformer for generating first and second beamformed signals responsive to sequential first and second transmit pulses, respectively, the first transmit pulse having one of a different phase and amplitude than the second transmit pulse;
an adder for combining of the first and second beamformed signals; and
a filter connected between the beamformer and the adder, the filter operable to filter the first and second beamformed signals to provide at least one of a frequency dependent amplitude weighting and frequency dependent phase shift to the first and second beamformed signals.
